(19) United States
(12) Patent Application Publication
(10) Pub. No.: US 2013/0304197 A1
(43) Pub. Date: Nov. 14, 2013

(54) CARDIAC VALVE MODIFICATION DEVICE

(71) Applicant: MValve Technologies Ltd., Herzliya (IL)

(72) Inventors: MAURICE BUCHBINDER, La Jolla, CA (US); AMIT TUBISHEVITZ, Tel Aviv (IL); SHAY DUBI, Tel Aviv (IL)

(21) Appl. No.: 13/779,326

(22) Filed: Feb. 27, 2013

Related U.S. Application Data
(60) Provisional application No. 61/604,103, filed on Feb. 28, 2012.
(60) Provisional application No. 61/604,083, filed on Feb. 28, 2012.

(51) Int. Cl. A61F 2/24

(52) U.S. Cl. CPC A61F 2/2427
USPC 623/2.11

(57) ABSTRACT

In an aspect, there is a prosthetic valve modification device adapted for endovascular delivery to a cardiac valve. The valve includes first and second support elements each having a collapsed delivery configuration and a deployed configuration. There are at least two bridging members extending from the first support element to the second support element, the bridging members having a delivery configuration and a deployed configuration. The bridging members either extend radially inward from the first and second support elements in the deployed configuration or are entirely straight and devoid of any visible curvature when in said deployed configuration.

### CROSS-REFERENCE TO PRIOR APPLICATIONS

The present application claims the benefit of U.S. Patent Application Nos. 61/604,103, filed on Feb. 28, 2012, and 61/604,083, filed on Feb. 28, 2012, the entire contents of each of which are incorporated herein by reference.

### BACKGROUND OF THE DISCLOSURE

Heart valve regurgitation occurs when the heart leaflets do not completely close when the heart contracts. When the heart contracts, blood flows back through the improperly closed leaflets. For example, mitral valve regurgitation occurs when blood flows back through the mitral valve and into the left atrium when the ventricle contracts.

In some instances regurgitation occurs due to disease of the valve leaflets (e.g., primary, or “organic” regurgitation). Regurgitation can also be caused by dilatation of the left ventricle, which can lead to secondary dilatation of the mitral valve annulus. Dilation of the annulus spreads the mitral valve leaflets apart and creates poor tip coaptation and secondary leakage, or so-called “functional regurgitation.”

Currently, primary regurgitation is corrected by attempting to remodel the native leaflets, such as with clips, sutures, hooks, etc., to allow them to close completely when the heart contracts. When the disease is too far advanced, the entire valve needs to be replaced with a prosthesis, either mechanical or biologic. Examples include suture annuloplasty rings all the way to actual valve replacement with leaflets, wherein the suture rings are sutured to the mitral valve annulus. Annuloplasty rings, which are also sutured to the annulus, have also been used to attempt to remodel the annulus, bringing the native leaflets closer together to allow them to properly close.

Based on the success of catheter-based aortic valve replacement there is growing interest in evaluating similar technologies to replace the mitral valve non-invasively using similar types of replacement valves.

Unlike the aortic valve, however, the mitral valve annulus does not provide a good landmark for positioning a replacement mitral valve. In patients needing a replacement aortic valve, the height and width of the aortic annulus are generally increased in the presence of degenerative disease associated with calcium formation. These changes in tissue make it easier to properly secure a replacement aortic valve in place due to the reduced cross-sectional area of the aortic annulus. The degenerative changes typically found in aortic valves are not, however, present in mitral valves experiencing regurgitation, and a mitral valve annulus is therefore generally thinner than the annulus of a diseased aortic valve. The thinner mitral valve annulus makes it relatively more difficult to properly seat a replacement mitral valve in the native mitral valve annulus. The general anatomy of the mitral valve annulus also makes it more difficult to properly anchor a replacement mitral valve in place. The mitral valve annulus provides for a smoother transition from the left atrium to the left ventricle than the transition that the aortic valve annulus provides from the aorta to the left ventricle. The aortic annulus is anatomically more pronounced, providing a larger “bump” to which a replacement aortic valve can more easily be secured in place.

In general, the aortic valve annulus is smaller than the mitral valve annulus. It has been estimated that the mitral valve annulus is about 2.4 cm to about 5 cm in diameter, while the aortic valve annulus has been estimated to be about 1.6 cm to about 2.5 cm in diameter.

A valve modification and support device is needed, that can be attached to an aortic replacement valve, either in the factory or in the OR/catheterization room prior to the procedure, and this modification-device enables implantation of the said aortic valve within the native mitral valve, thus modifying the aortic replacement valve into a mitral replacement valve. It should be noted that in this disclosure, the terms “valve modification device”, “valve modification and support device”, “valve support device”—are used interchangeably and refer to the same device of the invention

### SUMMARY OF THE INVENTION

One aspect of the disclosure is a valve-modification and support device, suitable for modifying a prosthetic aortic valve in order that it may be implanted and used as a replacement (prosthetic) mitral valve, such that after attachment of the modification device to the aortic replacement valve, said valve is readily implantable via endovascular delivery in a mitral position, said modification device comprising first and second support elements, wherein said first and second support elements each have a collapsed delivery configuration and a deployed configuration, and wherein at least two bridging members extend from the first support element to the second support element, said bridging members having a delivery configuration and a deployed configuration, wherein said bridging members either extend radially inward from the first and second support elements in the deployed configuration or are entirely straight and devoid of any visible curvature when in said deployed configuration.

In some embodiments the bridging members extend from discrete locations around adjacent support elements, and can be arranged symmetrically around the circumference of said support elements. Thus, in one embodiment, the first and second bridging members can extend from the adjacent support elements at points separated by about 180 degrees along the circumference of said support elements.

In certain other embodiments, the valve modification device may optionally further comprise secondary bridging members that mutually interconnect two or more main bridging members. In other embodiments, secondary bridging members are used to connect one or more of the main bridging members with the support elements. The term “secondary bridging members” is used in this context to distinguish said optional, additional bridges from the main bridging members that connect the first and second support elements, as disclosed hereinabove.

In another aspect, the prosthetic valve modification device comprises a single support element, wherein said support element has a collapsed delivery configuration and a deployed configuration. In one embodiment, the single support element is provided in the form of a flat annular ring, preferably constructed from a material having superelastic and/or shape memory properties. One example of such a suitable material is Nitinol, which possesses both of the aforementioned physical properties. These properties may be utilized in order to permit said device, following its delivery in a collapsed conformation, to return to an expanded memory configuration after being heated above its transition temperature. This embodiment of the modification device is also referred to herein as the ‘single-ring’ valve modification device, while the embodiment having two support elements connected by bridging members disclosed hereinabove, is also sometimes referred to as the ‘two-ring’ modification device.

In some embodiments at least one of the support elements (or the single support element in the case of the one-ring device) has an annular shape.

In some embodiments the bridging members and/or support elements are fitted with replacement valve engagement means adapted to securely engage a replacement heart valve. In some embodiment, the engagements means can have anchoring and/or locking elements adapted to securely lock with a portion of a replacement heart valve. In other embodiments, the replacement valve engagement means are formed from a soft biocompatible material (such as a biocompatible fabric, silicon, PET etc.) which are fitted to the external surface of portions of the support elements and/or bridging members. In these embodiments, the soft, compressible nature of the biocompatible material permits certain portions thereof to be compressed by the struts or other structural elements of the replacement valve, upon expansion within the lumen of the valve support. Other portions of the soft biocompatible material which are not compressed by the expanded replacement valve protrude into the internal space of said valve between the struts and/or other structural elements. The protrusions formed in this way engage and grip the replacement valve thereby preventing its movement in relation to the valve support. In other embodiments, the replacement valve engagement means comprise rigid anchors of a size and shape such that they are capable of entering the internal space of the replacement valve between its struts and/or other structural elements, upon expansion of said valve within the internal space of the valve support.

In some embodiments, the support elements and/or bridging members are fitted with heart tissue anchoring means adapted to securely anchor said support elements to the heart wall. Non-limiting examples of such anchoring means include hooks and spirals.

In some embodiments, the valve-modification device further comprises one or more stabilizing elements, the function of which is to provide additional stabilization of said support within the ventricle and/or atrium. Thus, in some embodiments, the valve-modification device comprises one or more intra-ventricular stabilizing elements, one or more intra-atrial stabilizing elements. In other embodiments, the cardiac valve support will be fitted with at least one intra-ventricular stabilizing element and at least one intra-atrial stabilizing element.

In some embodiments the support element(s) are adapted to preferentially bend at at least one location.

In some embodiments the support element(s) have a curved portion in their deployed configurations, wherein the curved portions are adapted to assume a tighter curved configuration in the collapsed delivery configurations.

In some embodiments of the two-ring modification device the first and second bridging members are generally C-shaped in their deployed configurations.

In some embodiments the support element has at least one coupling element adapted to reversibly couple to a delivery system. The at least one coupling element can be a threaded bore.

In some embodiments of the two-ring prosthetic valve modification device, the second support element has a dimension in the deployed configuration that is larger than a dimension of the first support element in the deployed configuration with or without one or more fixation elements attached and radially engaging in cardiac tissue when needed.

In some embodiments of the two-ring prosthetic valve modification device, the first and second support elements are connected by only two bridging members.

One aspect of the disclosure is a system adapted for endovascular or transapical delivery to replace a mitral valve, comprising: either a two-ring prosthetic valve modification device or a single-ring prosthetic valve modification device as disclosed hereinabove and a replacement heart valve comprising an expandable anchor and a plurality of leaflets adapted to be secured to the cardiac valve support. For the sake of clarity of description, the above disclosure of a delivery system comprising a two-ring prosthetic valve modification device relates to an embodiment of said device in which the two support elements are connected by two bridging members. However, it is to be recognized that the endovascular delivery system of the present invention may be used to deliver cardiac valve supports in which more than two bridging members mutually connect the two support elements.

In some embodiments the bridging members and/or support elements are adapted to securingly engage the replacement heart valve. In one such embodiment, the bridging members are formed such that at least one portion thereof comprises a series of folds or pleats (e.g. z-shaped pleats), the purpose of which is to increase the surface area of the bridging members that are available for interacting with the prosthetic replacement valve. An additional benefit of this embodiment is that the pleated region also assists in the transition between the delivery (closed) conformation of the valve modification device and the deployed (open) conformation thereof. In other embodiments, the replacement valve securing means comprise attachment means, such as hooks or other mechanical anchors that are connected, at one of their ends, to the support elements and/or bridging members, and have a free end for attachment to the replacement valve.

In some embodiments of the invention, the system disclosed hereinabove further comprises pressure measuring elements. These elements may be situated anywhere in the system—including on the surface of the valve modification device, attached to the replacement valve, as well as within the guide catheter. In another embodiment, the system of the invention further comprises connection terminals that permit the connection of pacemaker leads to various parts of said system.

One aspect of the disclosure is a method of replacing a patient's mitral valve, comprising: attaching a valve-modification device to an aortic replacement valve (either at the product manufacture or assembly site—e.g. in the factory—or in the hospital or other clinical setting prior to the procedure), the valve-modification device comprising a first support element a second support element, and at least two bridging members extending from the first and second support elements; Implanting the interconnected replacement valve and valve-modification device in the mitral valve annulus.

Similarly, the invention is also directed to a method of replacing a patient's mitral valve, comprising: the ex vivo attachment of a valve-modification device to an aortic replacement valve (either at the product manufacture or assembly site—e.g. in the factory—or in the hospital or other clinical treatment room prior to the procedure), the valve-modification device comprising a single support element; Implanting the interconnected replacement valve and valve-modification device in the mitral valve annulus.

In one embodiment, the above-defined methods may be employed to deliver the prosthetic valve and modifying device by an endovascular route. In another embodiment, the methods may be used to deliver the valve and modifying device by a transapical route.

The valve-modification device may be self expanding, or may be balloon expandable.

In a preferred embodiment the modifying device is self expandable and is constructed from biocompatible metals such as Nitinol, Cobalt based metal, Stainless steel.

In other embodiments, the above-defined method further comprises the step of causing intra-ventricular stabilizing elements and/or intra-atrial stabilizing elements to engage, respectively, the inner ventricular wall and/or inner atrial wall.

For the sake of clarity of description, the above disclosure of a method for replacing a patient's mitral valve using a two-ring prosthetic valve modification device relates to a method that uses a cardiac valve-modification device in which the two support elements are mutually connected by two bridging members. However, it is to be recognized that the endovascular delivery system of the present invention may be used to deliver cardiac valve supports containing more than two support elements and more than two bridging members.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C illustrate an exemplary replacement mitral valve support structure in an expanded configuration.

FIGS. 2A-2B illustrate a two-ring prosthetic valve modification device situated upon an exemplary stented-valve, wherein FIG. 2A shows the valve modification device mounted on stented-valve in an expanded position (post deployment), while FIG. 2B depicts the valve modification device mounted on a stented-valve in a pre-expanded position (pre-deployment).

FIG. 3 depicts and embodiment of a two-ring prosthetic valve modification device of the present invention, in which said device is constructed of a single wire.

FIG. 4 illustrates an embodiment of the lower support element of the presently-disclosed two-ring prosthetic valve modification device having a curved or cambered outer edge.

FIG. 5 depicts two support elements, each having the same internal diameter but different external diameters.

FIG. 6 illustrates an embodiment of a prosthetic valve modification device of the present invention fitted with two vertically-disposed stabilizing elements.

FIGS. 7A-7B depict embodiments of a prosthetic valve modification device, each having a stabilizing element formed from a stent-like mesh.

FIG. 8 illustrates an embodiment of the prosthetic valve modification device in which the stabilizing element contains spring-like constricted regions.

FIG. 9 illustrates an embodiment in which the prosthetic valve modification device is fitted with one horizontal stabilizing element and one vertical stabilizing element.

FIG. 10 depicts an embodiment of the prosthetic valve modification device having a plurality of stabilizing elements attached to the upper support element.

FIG. 11A-11C depict embodiments of the prosthetic valve modification device of the present invention in which the stabilizing elements are constructed in the form of curved arms.

FIG. 12 illustrates an embodiment of the prosthetic valve modification device of the present invention in which a horizontal ring-shaped stabilizing element is located beneath the single support element.

FIGS. 13A and 13B show a prosthetic valve modification device with a pair of elastic tab-like stabilizing elements attached to the upper support element.

FIG. 14 depicts a valve support containing spiral-shaped cardiac anchoring means.

FIG. 15 depicts a typical prosthetic valve modification device fitted with a number of hook-like anchors.

FIGS. 16A and 16B illustrate cardiac attachment anchors having backwardly pointing distal arms which may be retained in a closed position during delivery by means of a resorbable suture loop.

FIGS. 17A-17B illustrate two different embodiments of cover elements that may be used to conceal the cardiac attachment anchors during delivery of the prosthetic valve modification device.

FIGS. 18A-18B depict the use of a shape-memory anchor which is maintained in a straight conformation during delivery by means of an overtube.

FIG. 19 illustrates clip-like cardiac tissue anchors that are particularly suitable for attaching the support element to the annulus.

FIG. 20 illustrates a prosthetic valve modification device of the present invention fitted with two different types of valve engagement means.

FIGS. 21A and 21B illustrate support elements fitted with a valve engagement means constructed from a soft biocompatible material.

FIGS. 22A-22B depict an embodiment of the prosthetic valve modification device of the present invention in which said device includes flap components which are used to reduce para-valvular leakage.

### DETAILED DESCRIPTION OF PREFERRED EMBODIMENTS OF THE INVENTION

The disclosure is generally related to valve-modification devices that are adapted to be attached to a prosthetic aortic valve and thus allow its implantation near or within a native cardiac mitral valve or native mitral valve annulus.

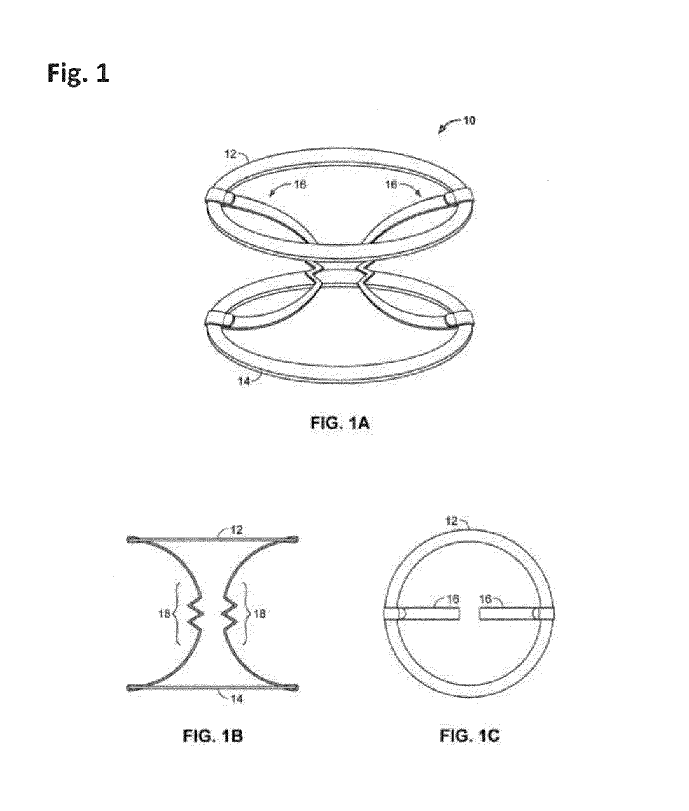

FIGS. 1A-1C illustrate an exemplary embodiment of a two-ring valve-modification device in an expanded configuration (Valve not shown). Valve modification device 10 includes a first support element 12, a second support element 14, and first and second bridge members 16 extending from first support 12 to second support 14. FIG. 1A illustrates a perspective view of valve modification device 10, while FIGS. 1B and 1C illustrate a side view and top-view, respectively, of valve modification device 10. As shown in FIG. 1B, each of bridge members 16 includes a valve engaging portion 18.

In some embodiments the first support element and the second support element are generally annular in shape in their expanded configurations (see, for example, FIG. 1A). Patient-to-patient variability in the cardiac anatomy can, however, require that the support elements have a variety of sizes and configurations. The support elements can therefore have any configuration as needed to be secured to any anatomical configuration. For example, the support elements can have generally elliptical configurations. Additionally, the support elements need not have the same general configuration. For example, the superior support element can have a generally annular shape and the inferior support element can have a generally elliptical shape. The bridge members operably connect the first and second support elements, and extend generally radially inward and axially away from a first of the support elements before extending radially outward towards the second of the support elements. For example, in the embodiment in FIGS. 1A-1C, bridge member 16 extends from support 12 in a radially inward direction and axially away from support element 12 and towards support element 14, before extending radially outward towards support 14. The valve engaging portions of the bridge members are disposed radially inward relative to the support elements. The bridge members are biased to the configurations shown in FIGS. 1A-1C, with the valve engaging portions disposed radially inward relative to the support elements. Because they are biased towards this configuration, they are adapted to apply a radially inward force to a subsequently positioned replacement mitral valve that is expanded to an expanded configuration within the bridge members (described below). The bridge members are therefore adapted to engage the replacement heart valve to secure the replacement mitral valve to the valve support.

In the embodiment in FIGS. 1A-1C, the bridge members extend from the support elements at discrete locations around the support elements. That is, in this embodiment, the bridge members do not extend from the support elements all the way around the support elements. If they did, the valve support would have a general hourglass shape. The bridge members, therefore, are not complete extensions of the support elements. While the embodiment in FIG. 1A-1C shows two bridge members extending from the support elements at discrete locations, the valve modification and support device may include more than two bridge members extending from the support elements at discrete locations along the support elements.

In the embodiment in FIGS. 1A-1C, the bridge members also symmetrically extend from the first and second support elements. That is, there is at least one line or plane extending through the valve that, in at least one view of the valve modification device, creates portions of the valve modification device that are symmetrical. For example, in reference to FIG. 1C, a line extending through and connecting the bridge members creates symmetrical portions of the valve modification device. Or, for example, in reference to FIG. 1B, a vertical line extending through the center of the valve modification device creates symmetrical portions of the valve modification device.

In some embodiments the first and second support elements and the bridge members are made from a resilient material that can be deformed into a delivery configuration yet are adapted to self-expand to an expanded configuration, with optional additional expansion of one or more components by balloon dilation. For example, the modification device can be made from Nitinol, relying on its superelastic properties. In some embodiments the valve modification device is made from a material with shape memory properties, such as nitinol, and is adapted to return to an expanded memory configuration after being heated above its transition temperature. In some embodiments in which the valve modification device is made from a material such as nitinol, the shape memory properties and the superelastic properties are utilized. In the embodiment in FIGS. 1A-1C, valve modification device 10 is adapted to return to the expanded configuration shown, either by self-expansion (relying on the superelasticity of the material), or by being heated above its transition temperature (such as by exposure to the body's temperature).

Expansion of the replacement mitral valve (e.g., balloon expansion, self-expansion, etc.) not only expands the replacement mitral valve, but applies an expanding force on the modification device bridge members, expanding them further radially outward towards the native annulus. Expansion of the replacement mitral valve causes the replacement valve and the modification device to engage the bridge members and secure the replacement mitral valve and modification device to the mitral annulus. Because the bridge members are biased towards a configuration in which they extend generally radially inward, the bridge members apply a radially inward force on the replacement mitral valve, helping to secure the replacement mitral valve in place.

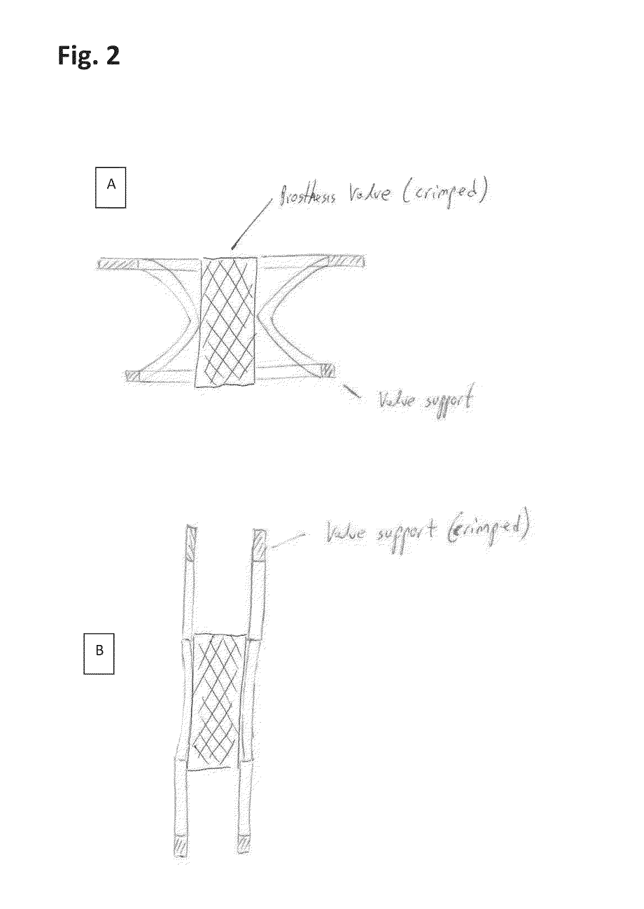

An illustration of a two-ring valve modification device situated upon an exemplary stented-valve is presented in FIG. 2.

FIG. 2A illustrates the valve modification device mounted on stented-valve in an expanded position, as would be after implantation in mitral annulus for example (post deployment).

FIG. 2B illustrates the valve modification device mounted on stented-valve in a pre-expanded position, as would be before deployment, during the time they are both situated within a catheter to be inserted into the body via endovascular method.

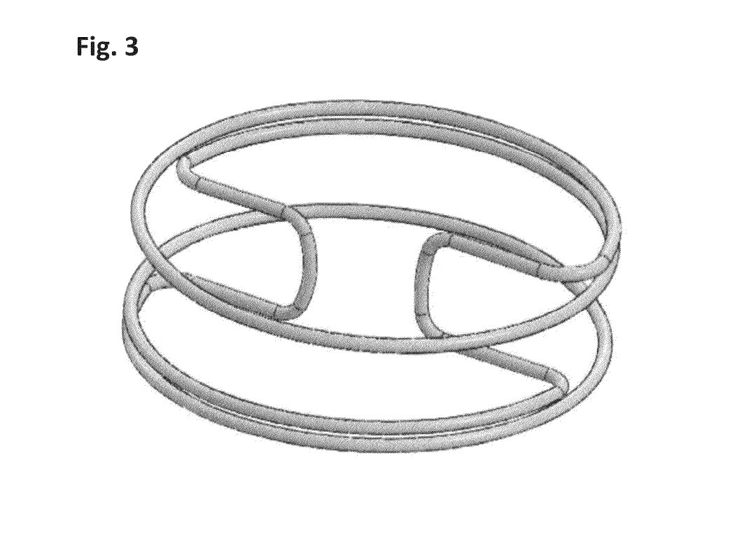

In the embodiment shown in FIGS. 1A-1C, the bridge members and support elements are separate and distinct elements secured to one another by any suitable technique (e.g., soldering). In some alternative embodiments, the support elements and the bridge members are manufactured as a single unit without components that need to be secured to one another. For example, in some embodiments the manufacturing of the valve modification device is simplified because it is manufactured from a single tubular shape memory material that is pre-formed with predetermined expansion ratios and forces needed to retain the replacement mitral valve in place. In some preferred embodiments of this type, the valve modification device is constructed of a single wire that has been shaped in a way to construct an upper support element, a lower support element, and two or more bridging elements between them. An example of an embodiment of this type is illustrated in FIG. 3. Suitable wire materials that may be used to manufacture valve supports of this type include (but are not limited to) biocompatible metals and metal alloys, Nitinol, cobalt and stainless steel. One advantage of this design is the fact that its simplicity of construction results in low manufacturing costs. A further significant advantage is that the use of a single wire (rather than a broader strip—as depicted in FIG. 1A) is that it may be collapsed to a very small size such that it may be inserted into a small diameter delivery catheter, thereby presenting a reduced crossing profile.

In some embodiments the height of the valve modification device, measured from the base of the first support to the top of the second support, is about 1 cm to about 5 cm to be able to accommodate the height of the replacement heart valve, such as a stented heart valve. In some embodiments the height is greater than 5 cm. In some embodiments the height of the valve modification device is between about 1 cm and about 2.5 cm. For example, a stented heart valve in an expanded configuration can have a height of about 17.5 mm. It should be noted, of course, that these numbers are merely exemplary and are not limiting in any way.

In some embodiments, the height of the two-ring valve modification device is less than the height of the replacement heart valve. Additionally, the two annular support elements can have different dimensions. For example, the two support elements, if generally annular-shaped, can have different diameters. In some embodiments the first support element has a larger diameter than the second support element because the anatomical position in which it is to be placed is larger than the anatomical position in which the second support element is to be placed. In the embodiment shown in FIGS. 1A-1C, support element 12 can have a larger diameter than support element 14 due to its expansion in the larger left atrium versus the smaller left ventricle, the papillary tendons and muscles, and other supporting structures in the left ventricle. The possible differences in dimensions of the superior and inferior support elements are discussed in more detail below.

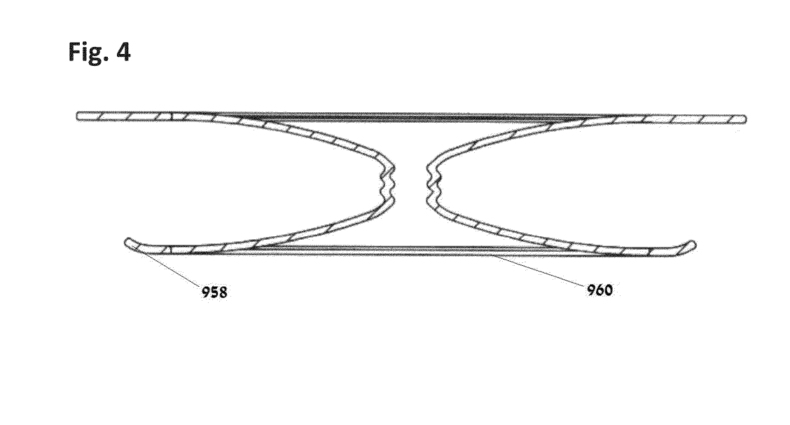

In other embodiments, the lower support element of the presently-disclosed valve modification device has a curved or cambered outer edge. An example of such an embodiment is shown in FIG. 4, in which the outer margins 958 of the lower support element 960 are seen to curve upwards. This type of embodiment is of particular value when the anatomical structure and size of the left ventricle is such that the valve modification device may interfere with the normal ejection of blood from said ventricle through the aortic valve during systole, for example by deflecting a portion of the blood away from the aortic valve.

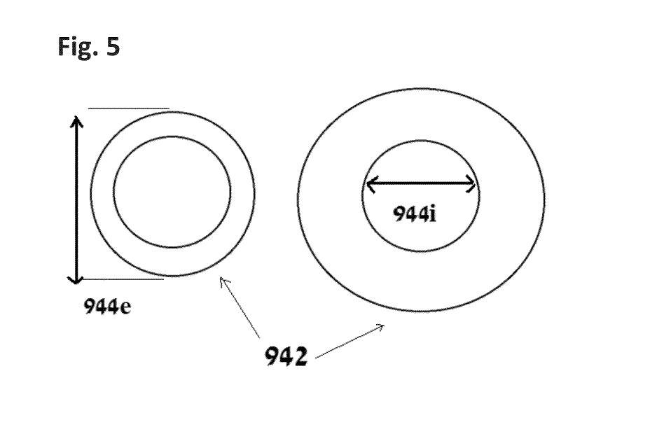

In most embodiments of both the single-ring and two-ring valve modification device disclosed herein, the sizes of the ring-like support elements may, as depicted in FIG. 5, be defined by two different dimensions—an external diameter 944e and an internal diameter 944i. It will be seen that while both of the support elements 942 shown in this figure have the same internal diameter, their external diameters differ. It will be appreciated that the internal diameter defines the space available for implantation of the replacement valve within the valve modification device, while the external diameter needs to be the same as the space within the native Mitral annulus (in order to permit stable implantation of the valve modification device). Since both the expanded diameter of different commercially-available replacement mitral valves and the diameter of the anatomical mitral annulus differs (from patient to patient), it follows that a range of valve modification devices needs to be manufactured and made available, such that the clinician can select the valve modification device having an internal diameter appropriate for the replacement valve to be implanted and an external diameter of the same size as the space within the mitral annulus.

In the embodiments described herein the support elements do not have a covering element. In some embodiments, however, one or more support elements can have a covering element such as a sealing skirt to enhance the sealing of blood flow in and around the support structure and replacement heart valve. The covering element can be any type of material that surrounds the support elements and provides the enhanced sealing functionality (e.g. it can prevent fluid leakage between the valve modification device and the heart wall). In some embodiments, the covering element can be attached (e.g. by the use of a biocompatible adhesive) to the outer surface of the support elements. In other embodiments, the covering element can be attached to the inner surface of the support elements.

In some embodiments one or more of support structures is covered in a material such as a polyester fabric (e.g., Dacron). Alternatively or in addition to, one or more of the bridge members can be covered in a polyester fabric such as Dacron.

In certain embodiments, the valve modification device (single ring or two-ring) may further comprise one or more stabilizing elements attached to the single ring, or in the case of the two-ring device, to the upper support element, the lower support element or to both of said elements. The purpose of the stabilizing elements is to increase the stability of the implanted valve modification device (and thus also enhance the stability of the implanted replacement valve), by means of stabilizing elements in the form of additional complete ring structures (in some cases, similar to the upper and lower support elements themselves), partial rings or curved arms, whereby said structures are placed such that at least part of their length is in close apposition to the surface of the inner ventricular wall and/or the surface of the inner atrial wall (in the case of stabilizing elements attached to the upper support element). Since the curvature of the inner walls of both the atrium and ventricle may be defined in relation to two mutually-perpendicular axes (horizontal and vertical), the stabilizing elements may be disposed either horizontally (i.e., essentially parallel to the horizontal axis of the valve modification device) or vertically (i.e. essentially parallel to the vertical axis of the valve modification device). Additionally, in some embodiments, the stabilizing elements may be disposed such that they are neither parallel to the horizontal axis nor to the vertical axis, but rather are arranged at an acute angle to one of these axes.

In some cases, the stabilizing elements (which may be formed from either elastic or plastic materials, as will be described hereinbelow) will be manufactured as an integral part of the valve modification device. In other cases, said stabilizing elements will be manufactured separately (by casting, milling, laser-cutting or any other suitable technique known to skilled artisans in the field), and later connected to one or both support elements by means of soldering or laser welding.

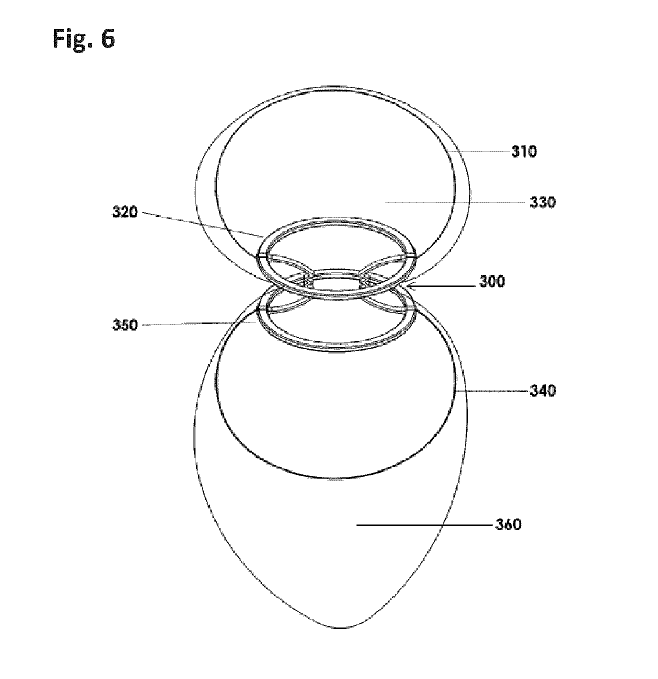

FIG. 6 illustrates a valve modification device 300 of the present invention fitted with two vertically-disposed ring-shaped stabilizing elements. As shown in the figure, the upper, apical ring 310 is attached at its lower portion to the upper support element 320, while its upper portion is disposed within the atrium 330, in close contact with the inner atrial wall. Conversely, the lower, ventricular ring 340 is attached, at its upper end, to the lower support element 350, while its lower portion is disposed within the ventricle 360, in close contact with the inner ventricular wall.

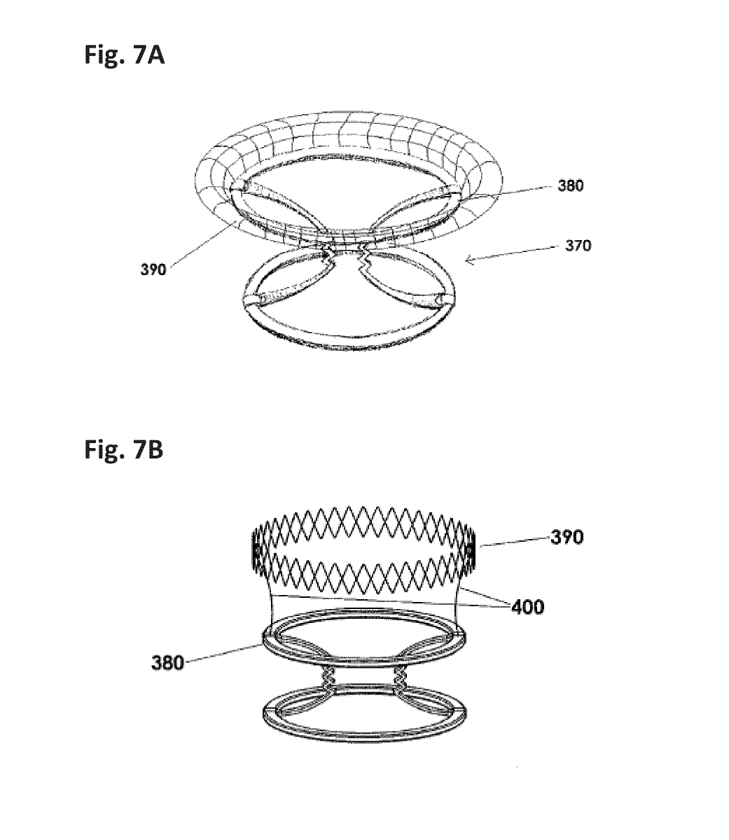

In the case of horizontal stabilizing elements, the element itself can (as explained above) be a complete ring, a partial ring or a curved elongate arm. While in some complete ring embodiments (as shown, for example, in FIG. 6), the stabilizing element is constructed from a single looped wire or solid band, in other embodiments, it may be constructed in the form of a stent-like mesh. FIG. 7A illustrates one embodiment of this type, in which the mesh-like stabilizing element 390 is attached directly to the upper support element 380 of valve modification device 370. Alternatively, as shown in FIG. 7B, the mesh-like stabilizing element 390 may be connected to the upper support element 380 by means of additional bridging members 400, which serve as spacer arms, increasing the separation distance between the stent-like mesh stabilizer 390 and said support element 380.

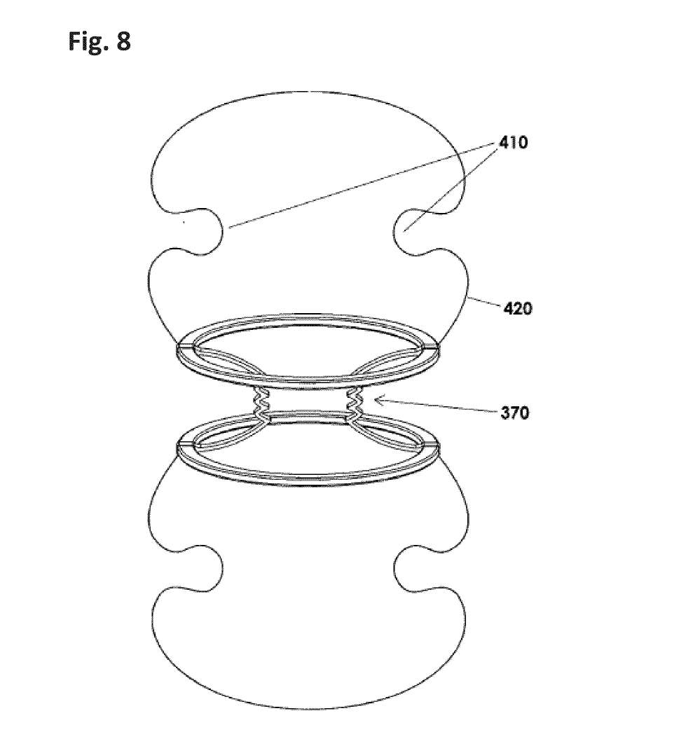

While the stabilizing element is generally constructed such that its outline shape is that of a smooth curve, in one preferred embodiment, as depicted in FIG. 8, this smooth curve is broken by one or more constricted regions 410, wherein said regions act as spring-like elements, increasing the force that said stabilizing element 420 is capable of applying to the inner ventricular or atrial wall, and thereby enhancing the ability of said stabilizing element to stabilize the valve modification device 370. The device shown in FIG. 8 contains two vertical stabilizing elements—a ventricular stabilizing element attached to the lower support element and an atrial stabilizing element attached to the upper support element. In other versions of this embodiment, the valve modification device may be fitted with one vertical stabilizing element (attached to one support element) and one horizontal stabilizing element (attached to the other support element). In some other embodiments, the valve modification device contains only one such stabilizing element (horizontal, vertical or otherwise angled). In still further embodiments, a single valve modification device may contain one stabilizing element containing one or more constricted regions 410, as shown in FIG. 8, together with one or more stabilizing elements of any of the other types disclosed and described herein.

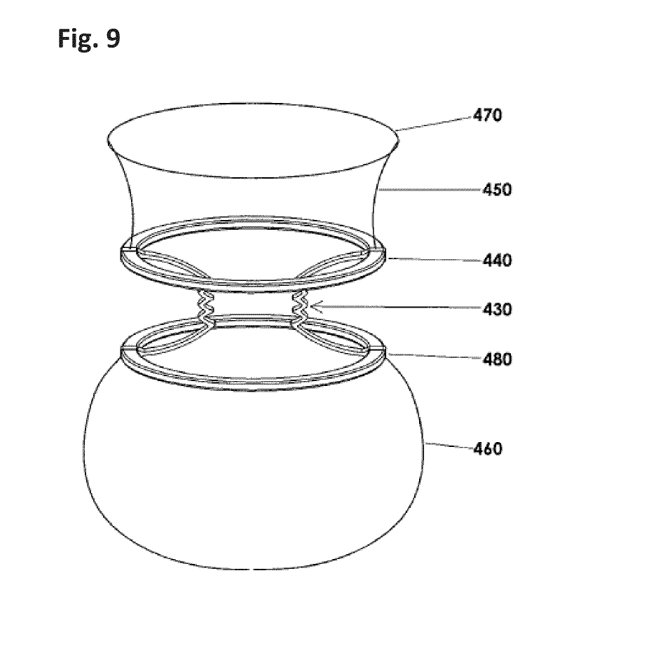

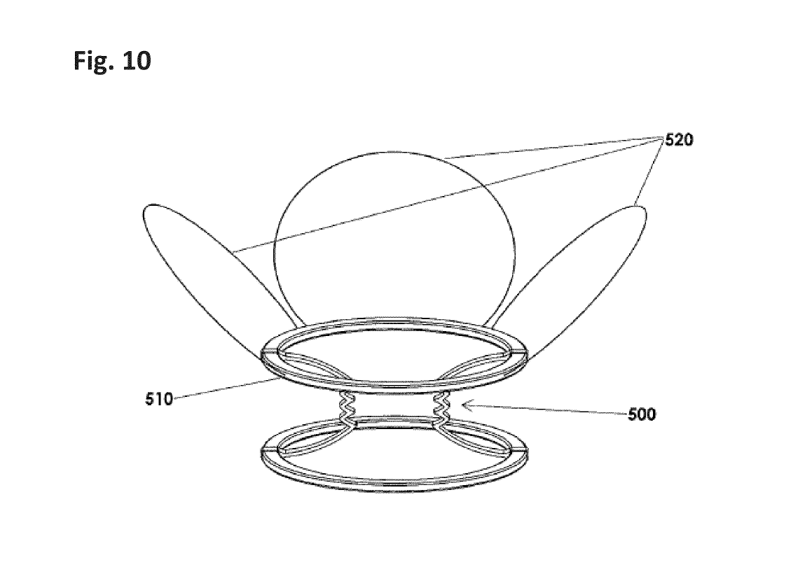

A further example of a valve modification device fitted with a combination of different stabilizing elements is shown in FIG. 9. Thus, lower support element 480 of valve modification device 430 is fitted with a vertically aligned ring-like ventricular stabilizing element 460, while a horizontally-aligned atrial stabilizing element 470 is connected via additional bridging elements 450 to upper support element 440. While only two additional bridging elements 450 are depicted in this figure, as many such elements as necessary may be incorporated into the device. Of course, in other versions, the arrangement of the stabilizing elements shown in FIG. 9 may be reversed, such that the valve modification device contains a horizontal lower stabilizing element and a vertical upper stabilizing element. As mentioned above, all possible combinations of the various types of stabilizing element disclosed herein may be used, as appropriate. It should also be noted that more than one stabilizing element may be attached to one or both support elements. FIG. 10 illustrates one embodiment of this type, in which the upper support element 510 of the valve modification device 500 is fitted with several (in this case, three) non-horizontal, angled, atrial stabilizing elements 520. It is to be emphasized that in all of the embodiments illustrated in FIGS. 1-10 that were discussed hereinabove, single-ring valve modification devices may be used interchangeably with the two-ring devices depicted in said figures.

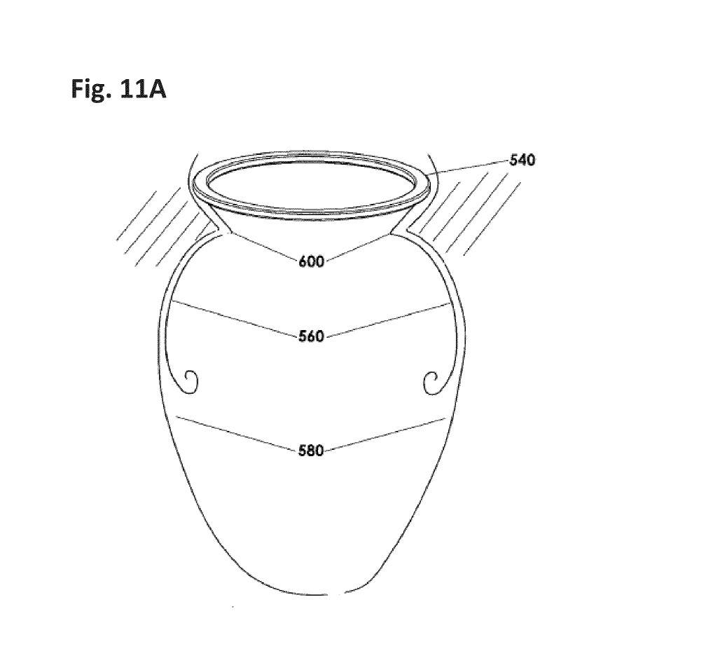

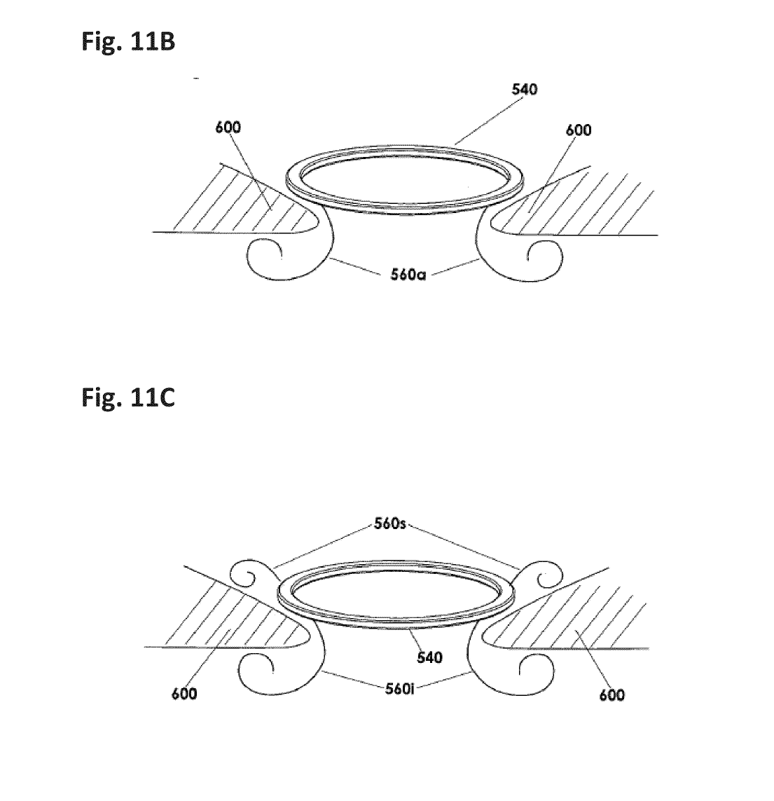

As explained hereinabove, the stabilizing element need not be provided in the form of a complete ring, but rather may also have the form of a partial ring or a curved elongate arm. Various examples of the latter type of stabilizing element are shown in FIGS. 11A, B and C, which illustrate the use of a single-ring valve modification device. The particular forms of the stabilizing element shown in these figures may, of course, equally be used in conjunction with a two-ring valve modification device, in which said stabilizing elements may be connected to the upper support ring only, the lower ring only or to both support rings. Thus, FIG. 11A depicts a support element 540 of a single-ring valve modification device of the present invention, wherein said valve modification device is connected to—and stabilized by—two curved elongate arms 560 which are disposed vertically downwards along the inner ventricular wall 580. In the example shown in this figure, the stabilizing elements 560 are constructed from an elastic material (such as cobalt base alloy, nitinol, stainless steel and other biocompatible metals and metal alloys). The curved arms typically have a length of between 1 mm and 50 mm, preferably about 20 mm. As will be seen in the figure, the upper part of each stabilizing element 560 is angled such that it is able to pass around the cardiac annulus 600. In some embodiments, the elongate, curved elastic arms may be constructed such that they are in a state of pre-load. The elastic properties of the stabilizing elements will cause said element to tend to both grip the annulus and to apply an outward force on the ventricular wall inferior to the annulus. In an alternative embodiment of this aspect of the invention, the curved elongate stabilizing elements may be constructed from a plastically-deformable material such as stainless steel, cobalt base alloy and nitinol. In this case, the elongate arms are molded around the annulus using a clenching or crimping tool. In this way, the upper sections of the elongate arms will firmly grip the annulus, while the lower sections will be biased outward and downwards along the ventricular wall.

FIG. 11B illustrates another embodiment of this aspect of the device, wherein the stabilizing elements 560a attached to the single support element 540 are much shorter than those shown in FIG. 11A, and apply a stabilizing force to the inferior surface of the annulus 600 (rather than to the lateral inner walls of the ventricle). During implantation, the stabilizing elements are brought into position below the annulus, such that the annulus becomes “trapped” between said stabilizing elements and the support element itself.

A still further variant of this embodiment is illustrated in FIG. 11C. This variant differs from the embodiment shown in FIG. 11B, in that the single support element 540 is fitted with both upper (560s) and lower (560i) stabilizing elements. During implantation into a patient, the valve modification device is manipulated such that the annulus 600 becomes “trapped” between these upper and lower stabilizing elements. In each of the variants of this embodiment, the short stabilizing elements may be brought into position by means of a balloon expansion mechanism, by a mechanical closure mechanism or, alternatively, said stabilizing elements may be self-expanding.

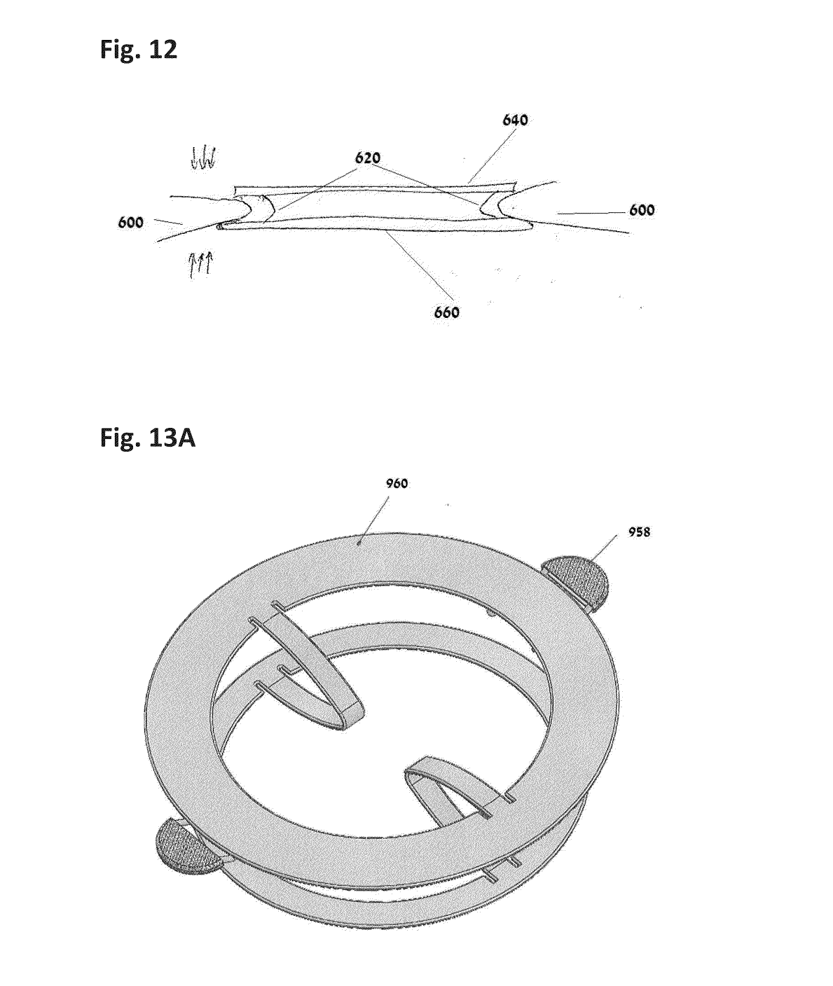

FIG. 12 depicts an alternative design of the valve modification device of the present invention, additionally comprising a horizontally-disposed ring-shaped stabilizing element 660, located beneath the single support element 640. Elastic members 620 mutually connect the support element (640) and said additional ring support (660). The annulus 600 may thus become trapped or pinched between them (as indicated by the arrows). This design may either be used without any additional stabilization elements, or in combination with any of the stabilization element embodiments described hereinabove. It is to be noted that although FIG. 12 depicts the use of this type of stabilization element in conjunction with a single-ring valve modification device, it may equally be used with a two-ring modification device.

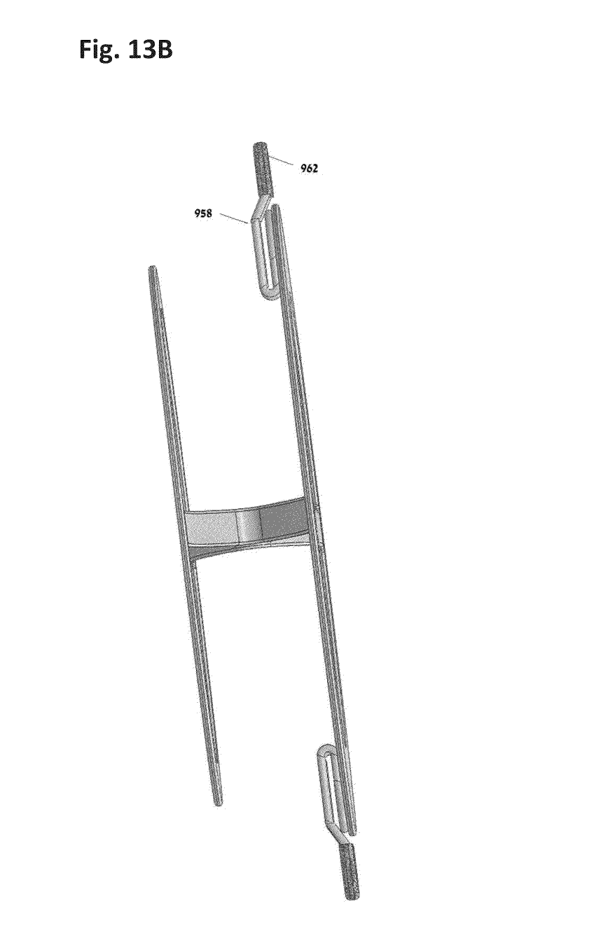

In a still further embodiment, as depicted in FIG. 13A, a two-ring valve modification device as viewed from above is seen to comprise a pair of elastic stabilizing elements 958, one on each side of the upper support element 960. These stabilizing elements may be manufactured from biocompatible metals including (but not limited to) Nitinol, Cobalt and Stainless steel, and are manufactured in the form of a spring-like tab that permits the elastic forces applied by the device on the ventricular wall to be distributed over a large surface area, so as to minimize local pressure on the cardiac tissue, thus minimizing the danger of necrosis of cardiac tissue due to high-level mechanical stress. The structure of the tab-like stabilizing elements 958 may be better seen in the side view of this embodiment of the device, presented in FIG. 13B. As may be seen from these figures, each tab may preferably be covered by a biocompatible fabric or mesh 962 (for example made from Dacron, PTFE etc.), the key functions of which are to assist in distributing the force, as previously explained, and also to encourage growth of cardiac tissue on the device, thus improving the attachment thereof to the heart wall. One particular advantage of using this type of stabilizing element is that it approximates the upper support element to the floor of the left atrium, thus essentially compressing the annulus (the stabilizing element compressing from the ventricular side and the upper support element compressing from the atrial side), thereby forming a “plug” that will prevent paravalvular leakage, even in cases in which the annulus is larger in diameter than the prosthetic valve, provided that the upper support element is larger than the annulus. In this embodiment, the upper support element may be fitted with one or more stabilizing elements of this type, which may be distributed evenly or unevenly around the circumference of said support element. Exemplary dimensions of this tab-like stabilizing element are as follows: width 2-20 mm; and length 2-20 mm. However, it is to be recognized that these measurements are for the purposes of illustration only, stabilizing elements of dimensions larger or smaller than these ranges being included within the scope of the present invention.

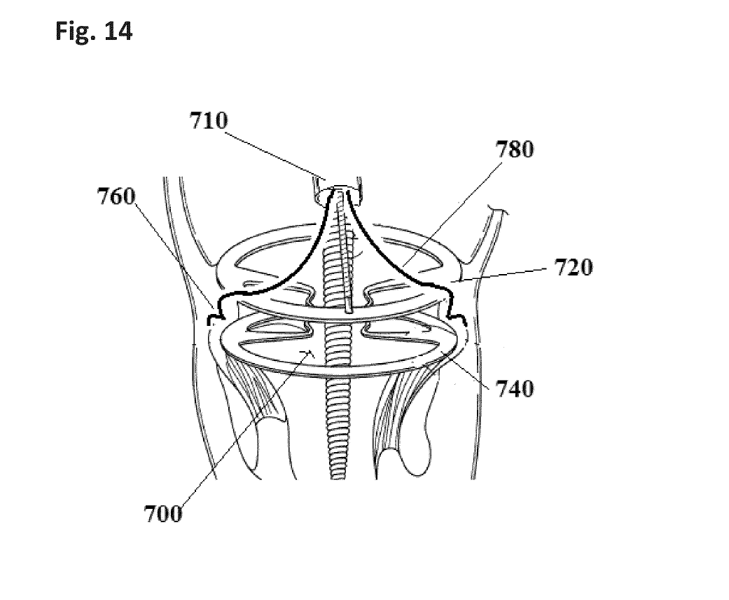

In some embodiments of the present invention, the careful selection of a correctly-sized valve modification device will permit said modification device to be self retaining in the region of the annulus following self-expansion during device delivery, as will be described hereinbelow. In other cases, however, the valve modification device of the present invention will further comprise one or more heart tissue anchoring means or mechanisms (connected to the support elements and/or bridging members) for firmly anchoring said valve modification device to the cardiac tissue. In one embodiment of this aspect, the cardiac anchoring means comprise a plurality of spiral or hook-like anchors. An example of this type of anchoring means is illustrated in FIG. 14, which shows a guide catheter 710 being used to deliver a valve modification device 700 of the present invention. At the stage of the delivery process shown in this figure (which will be described in more detail hereinbelow), both of the support elements, 720 and 740, as well as the bridging members have self-expanded into their working conformations. It will be seen that the upper support element is fitted with two spiral cardiac attachment anchors 760, the sharp free ends of which face laterally. The bases (i.e. medial ends) of the anchors are connected to control wires 780 that pass upwards and proximally through guide catheter 710, eventually leaving the patient's body and ending at a proximal control console. Once the valve modification device has been manipulated into the desired position (as shown in the figure), the spiral anchors 760 are caused to rotate by means of the operator manipulating the proximal ends of the control wires, thereby becoming inserted within the cardiac tissue and thus firmly anchoring the valve modification device in its operating position.

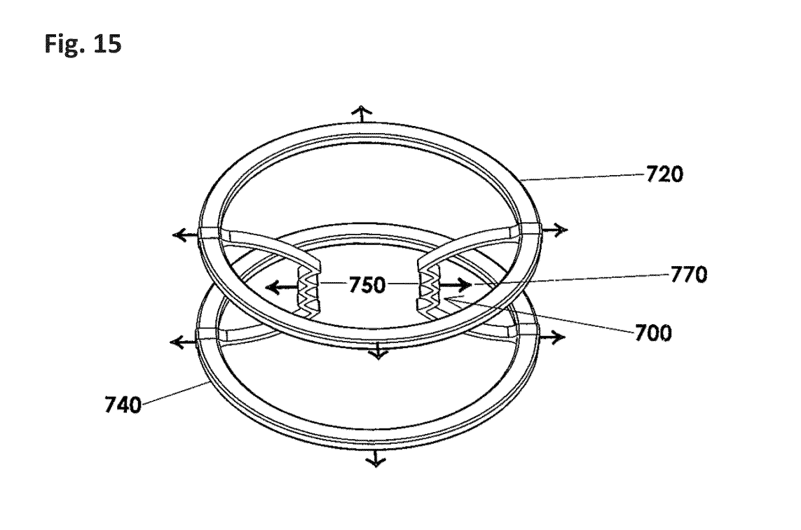

It is to be noted that FIG. 14 presents only one exemplary design for the cardiac tissue anchors, and many others are possible and included within the scope of the present invention. Thus, in another embodiment, hook-like anchors are attached at various points along the surface of the valve modification device, either on the support elements, the bridging members or both. This embodiment is illustrated in FIG. 15 which depicts a typical valve modification device 700, comprising an upper support element 720, a lower support element 740 and two bridging members 750, on the surface of all of which are distributed a number of hook-like anchors 770. (Nine such anchors are shown in the figure.)

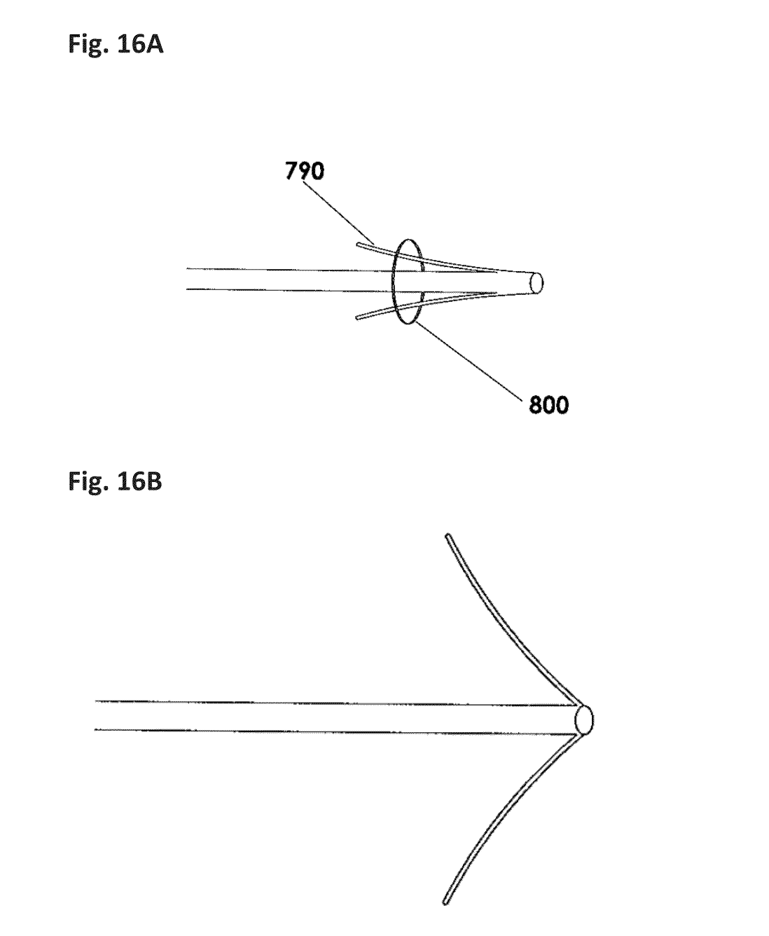

In some situations, it is advantageous for the cardiac tissue anchors to adopt a closed, inactive conformation during insertion of the valve modification device into the body, in order to avoid both trauma to the patients tissues and to avoid premature anchoring (for example at an incorrect location). Then, when said device is correctly positioned, the anchors would be caused to move from their closed, inactive conformation to an open active position. There are a number of ways to implement this type of embodiment. Thus, in a first implementation, the cardiac attachment anchor is constructed with two or more backwardly-pointing self-opening distal arms. During insertion and implantation, the distal arms are retained in a closed conformation by means of a small loop of resorbable suture material. Then, after a certain period of time following insertion of said attachment means into the ventricular tissue (e.g. between a few hours and a few weeks), said suture dissolves, thereby permitting the distal arms to adopt their open conformation. This embodiment is illustrated in FIGS. 16A and 16B: in FIG. 16A, the distal anchor arms 790 are shown retained in their closed position by means of suture 800. In FIG. 16B, the required length of time has elapsed (following insertion) and the suture has dissolved, releasing the distal anchor arms and allowing them to spread apart within the cardiac tissue, thereby increasing the resistance to withdrawal offered by said anchor.

In a further embodiment of this type, the anchor hooks are manufactured from a shape memory material, such as biocompatible nickel-titanium alloys (e.g. Nitinol). During insertion, the anchors are in their closed conformation, but following the implantation procedure the rise in temperature experienced during insertion into the patient's body results in opening of the anchors, as they regain their initial shape.

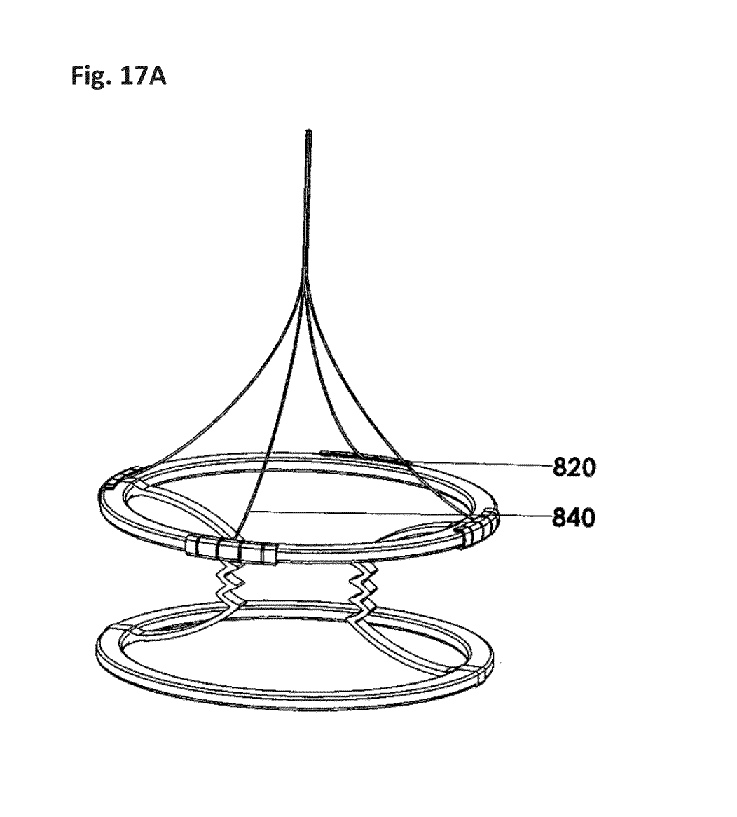

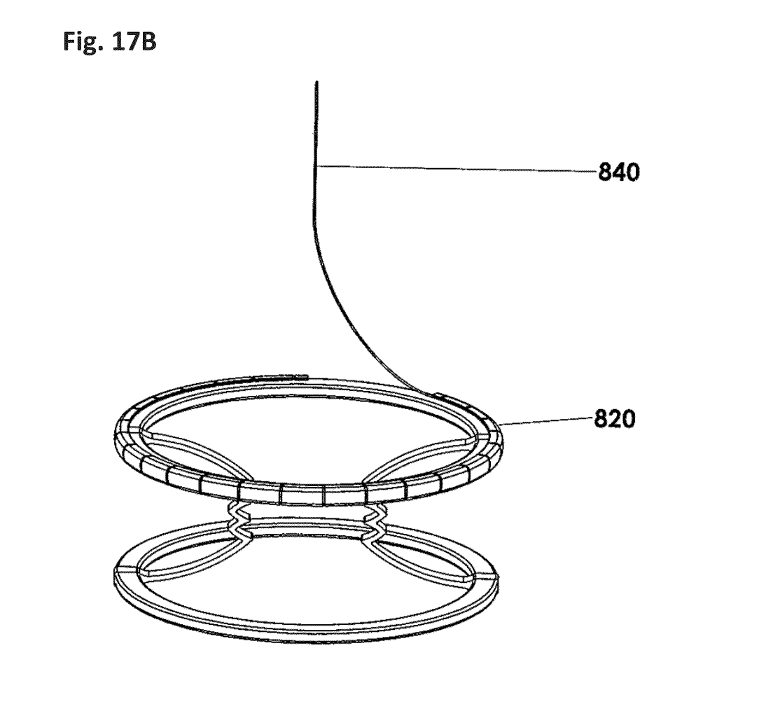

In a still further embodiment of this type, as shown in FIGS. 17A and 17B, the anchor hooks are protected by a cover element 820 (such as a sleeve or a piece of tubing) which is manufactured from a material with limited flexibility, such as PET, nylon and similar biocompatible plastics. After the operator is satisfied that the valve modification device has been implanted at the correct site, control elements 840 attached to the cover elements are pulled, thereby withdrawing them through the guide catheter, thus permitting the anchor hooks to freely adopt their open conformation and to become inserted into the cardiac tissue. In the design shown in FIG. 17A, each anchor is protected by its own individual cover, while in FIG. 17B a single cover element protects all of the anchors (not shown) that are attached to the upper support element.

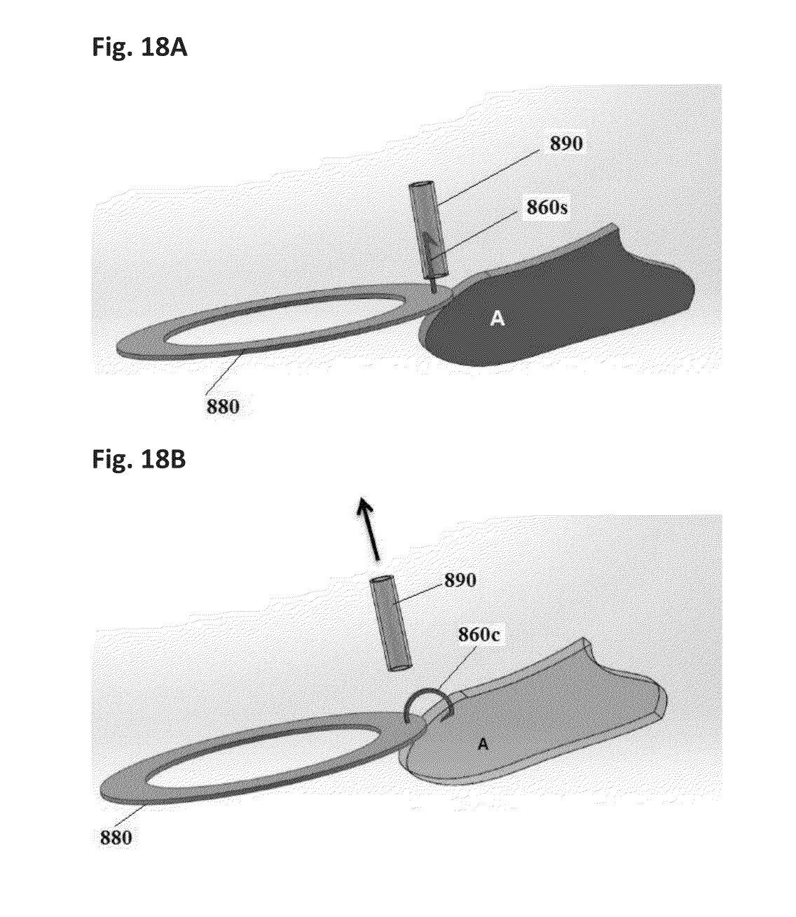

FIGS. 18A and 18B illustrate a yet further embodiment of this aspect of the invention. Thus FIG. 18A shows a barbed anchor 860s attached to a support element 880 is maintained in an inactive, straight conformation by means of an overtube 890, which also serves to protect the patient's tissues from trauma during insertion and implantation of the valve modification device. Following implantation at the desired site, as shown in FIG. 18B, overtube 890 is pulled away from the anchor 860c (for example, by means of pulling a control wire), which now adopts its “natural”, curved conformation, during which shape transition, said anchor now pierces the cardiac tissue (indicated by the letter A in the figure). Suitable anchors for use in this embodiment can be manufactured from shape-memory materials or from super-elasticity materials such as Nitinol, cobalt base alloy and spring-tempered stainless steel. Typically, anchors of this type will have a mid-length diameter of between about 0.2 mm and 1 mm, and a length in the range of about 2 to about 10 mm. Suitable overtubes may be manufactured from biocompatible polymers such as braided nylon and PET to a tolerance that permits a tight fit over the anchor.

It is to be noted that the cardiac tissue anchors described hereinabove may, in certain cases, be used to attach the valve modification device of the present invention to the anatomical valve leaflets and chordae (in addition to, or instead of attaching said device to the inner ventricular wall). In this regard, the present invention also encompasses additional types of cardiac tissue anchor which are characterized by having a plurality of anchoring wires that advantageously become entangled within the valve leaflets and chordae. Anchors of this type are particularly suitable for use in attaching the lower support element and bridging members to the aforementioned anatomical structures.

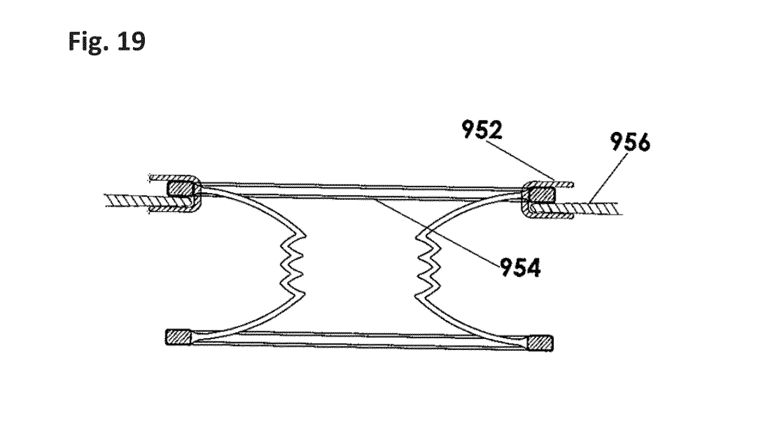

In one still further embodiment, the cardiac tissue anchors may be provided in the form of small clips (similar to vascular clips used to close blood vessels during surgical procedures, and well known to the skilled artisan). An example of the use of this embodiment is shown in FIG. 19, in which clip 952 is used to attach the upper support element 954 to the annulus 956. Clips of this type may also be used to attach the upper support element to atrial wall tissue and/or anatomical valve leaflets. In one particularly preferred embodiment the clip is caused to attach to the tissue in the area of the trigone—an anatomical area, on two opposite sides of the mitral valve, which has more fibrous tissue—and which is therefore able to provide a firm base for anchoring the valve modification device.

In another embodiment (not shown), the clip may be an integral part of the upper or lower rings, or the bridges. This may be achieved by attaching one of the jaws of the clip to the valve modification device, while the second of the jaws is free to be plastically deformed and to become anchored to the tissue.

In the case of certain replacement valves that may be used in conjunction with the valve modification device of the present invention, the radially-outward forces exerted by the expanded replacement valve are sufficient to stably retain said valve within the inner cavity of said valve modification device. However, in some instances—particularly when self-expanding replacement valves are being implanted—the radial force exerted by the expanded valve may be insufficient to ensure that it can withstand all of the physiological forces exerted therein during all stages of the cardiac cycle. In such circumstances, the bridging members and/or support elements of the valve modification device may further comprise a valve engagement portion. In one embodiment, the valve engagement portion may comprise a series of zigzag-like folds or pleats in the central, innermost region of the bridging members. These folds or pleats interact with the struts or other structural features of the replacement valve, thereby stabilizing said valve within the valve modification device.

In another embodiment, the valve engagement means comprise either inward facing or outward facing anchors, whose purpose is engage with the external struts of the replacement valve, thereby stabilizing said valve within the modification device.

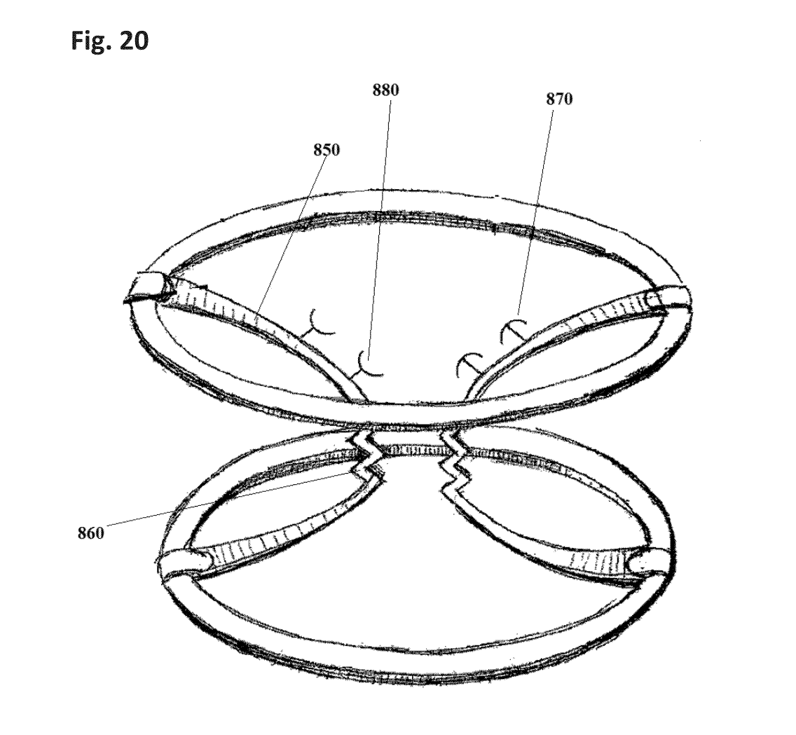

FIG. 20 depicts a valve modification device of the present invention comprising both of the aforementioned embodiments of valve engagement means. Thus, it may be seen that the central portions of the bridging members 850 are folded into a series of pleats 860. In addition, said bridging members are also provided with both inward facing 870 and outward facing 880 anchors.

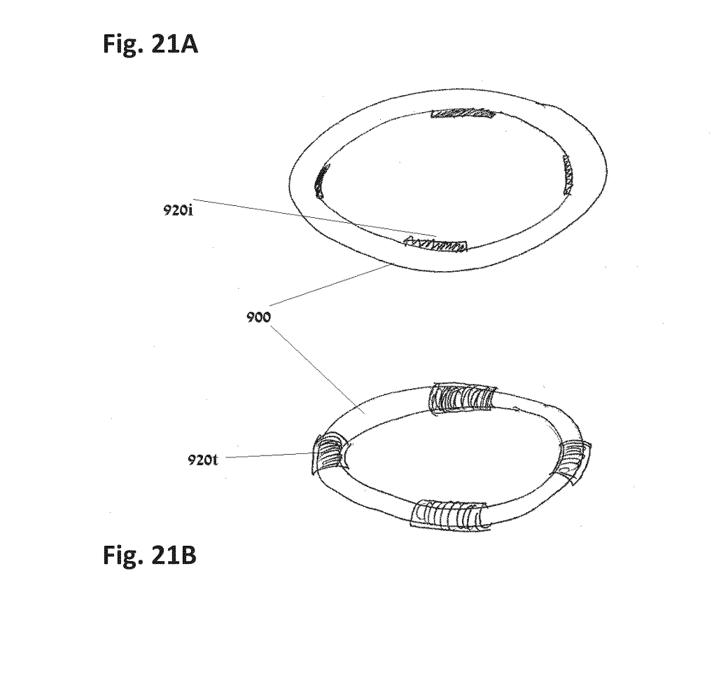

FIGS. 21A and 21B show a still further embodiment of the valve engagement means, attached to an exemplary support element 900 of the present invention. Thus, in FIG. 21A, four short lengths of a soft biocompatible material (such as a biocompatible fabric, silicon, PET etc.) 920i are attached to the inner surface of element 900. Upon expansion of the replacement valve stent within the inner space of the valve modification device, the soft material is caused to penetrate between the valve stent struts, thereby forming engagement “teeth” that serve to stabilize the replacement valve—support device assembly. FIG. 21B depicts a very similar set of four valve engagement means 920t formed from a soft biocompatible material. However, in the case of this version, the soft material is provided in the form of tubular sleeves surrounding (partially or completely) support element 900 at the four locations shown in the figure.

A delivery device appropriate for the valve modification device of this invention and suitable for endoscopic delivery was disclosed in a co-owned, co-pending U.S. application (Ser. No. 13/224,124, filed on Sep. 1, 2011 and published as US 2012/0059458). The said delivery device may be used for trans-septal or trans-apical delivery of the replacement valve and valve-modification device.

Access to the mitral valve or other atrioventricular valve will preferably be accomplished through the patient's vasculature percutaneously (access through the skin). Percutaneous access to a remote vasculature location is well-known in the art. Depending on the point of vascular access, the approach to the mitral valve can be antegrade and require entry into the left atrium by crossing the interatrial septum. Alternatively, approach to the mitral valve may be retrograde where the left ventricle is entered through the aortic valve. Alternatively, the mitral valve can be accessed transapically, a procedure known in the art. Additional details of an exemplary antegrade approach through the interatrial septum and other suitable access approaches can be found in the art, such as in U.S. Pat. No. 7,753,923, filed Aug. 25, 2004, the contents of which are incorporated herein by reference.

While the support structures herein are generally described as a support for prosthetic valves for use in the mitral annulus, they can be delivered to a desired location to support other replacement cardiac valves, such as replacement tricuspid valves, replacement pulmonic valves, and replacement aortic valves.

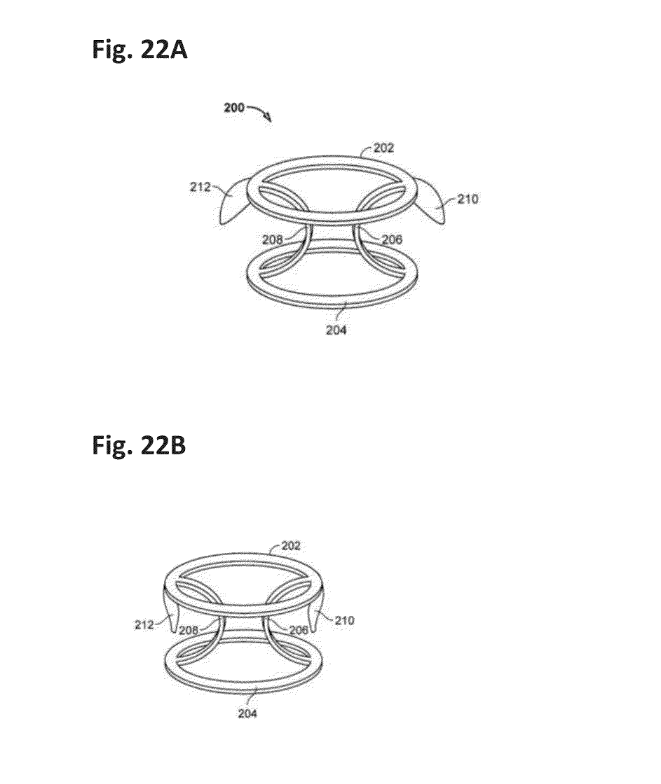

FIGS. 22A and 22B illustrate an alternative embodiment of a valve modification device. Valve modification device 200 includes components to mitigate para-valvular leakage. In addition to support elements 202 and 204 and bridge members 206 and 208, valve modification device 200 includes one or more flaps 210 and 212. The flaps extend coverage of the valve modification device system and help mitigate para-valvular leakage, functioning similarly to mudflaps on an automobile. During delivery exemplary flaps 210 and 212 are tucked around or against superior support element 202 as shown in FIG. 22B, and upon deployment from the catheter, flaps expand or extend to the configuration shown in FIG. 22A (native valve not shown for clarity). The flaps can be made of a flexible biocompatible material such as a wide variety of polymeric compositions. The flaps can be secured to the valve modification device by any suitable mechanism, such as by suturing the flaps to the support element, or to covered material, and using the bridge member to prevent the suture material from being displaced.

When deployed, in some embodiments the flaps are disposed above the annulus and over the side of the superior support element, which may not be extending all the way to the atrial wall. This can extend coverage of the valve modification device system for a few millimeters, reducing para-valvular leakage. Alternatively, in some embodiments in which the support element is larger, the flaps are urged against the atrial tissue. In this use, the flaps act as an additional seal when the valve modification device system is in place. The one or more flaps can therefore be a component of the valve modification device system that reduces para-valvular leakage and/or acts as an additional seal.

As explained hereinabove, in a highly preferred embodiment of the present invention, the valve modification device is used to modify a prosthetic aortic valve such that it may be implanted in the mitral valve annulus. Any suitable commercially available prosthetic aortic valve may be used to work the present invention, including both balloon-expandable and self-expanding valves. Examples include (but are not limited to): Sapien Valve (Edwards Lifesciences Inc., US), Lotus Valve (Boston Scientific Inc., US), CoreValve (Medtronic Inc.) and DFM valve (Direct Flow Medical Inc., US).

While some embodiments have been shown and described herein, it will be obvious to those skilled in the art that such embodiments are provided by way of example only. In particular, it is to be recognized that all of the embodiments employing two-ring valve modification devices shown in the accompanying figures may also be implemented using single-ring modification devices, and vice versa. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A prosthetic valve modification device adapted for endovascular delivery to a cardiac valve, comprising:
first and second support elements each having a collapsed delivery configuration and a deployed configuration;
and wherein at least two bridging members extend from the first support element to the second support element, said bridging members having a delivery configuration and a deployed m configuration, wherein said bridging members either extend radially inward from the first and second support elements in the deployed configuration, or are entirely straight and devoid of any visible curvature when in said deployed configuration.

2. A prosthetic valve modification device according to claim 1, wherein said device further comprises one or more atrial and/or ventricular stabilization elements.

3. A prosthetic valve modification device adapted for endovascular delivery to a cardiac valve, comprising:
a single support element having a collapsed delivery configuration and a deployed configuration.

4. A prosthetic valve modification device according to claim 3, wherein said device further comprises one or more atrial and/or ventricular stabilization elements.

5. A system adapted for endovascular delivery or transapical delivery to replace a mitral valve, comprising:
a cardiac valve modification device according to any one of the previous claims; and
a prosthetic heart valve comprising an expandable anchor and a plurality of leaflets adapted to be secured to the cardiac valve modification device.

6. A system according to claim 5, wherein the prosthetic heart valve is a prosthetic aortic valve.

7. A method for replacing a patient's mitral valve, comprising attaching a valve-modification device to an aortic replacement valve prior to the clinical procedure, the valve-modification device comprising a first support element a second support element, and at least two bridging members extending from the first and second support elements; and implanting the interconnected replacement valve and valve-modification device in the mitral valve annulus.

8. The method according to claim 7, wherein the attachment of the valve-modification device to an aortic replacement valve occurs at the product manufacture or assembly site.

9. The method according to claim 7, wherein the attachment of the valve-modification device to an aortic replacement valve occurs in the clinical treatment room.

10. A method for replacing a patient's mitral valve, comprising attaching a valve-modification device to an aortic replacement valve prior to the clinical procedure, the valve-modification device comprising a single support element; implanting the interconnected replacement valve and valve-modification device in the mitral valve annulus.

11. The method according to claim 7, wherein the attachment of the valve-modification device to an aortic replacement valve occurs at the product manufacture or assembly site.

12. The method according to claim 7, wherein the attachment of the valve-modification device to an aortic replacement valve occurs in the clinical treatment room.
